(19) United States
(12) Patent Application Publication
(10) Pub. No.: US 2014/0100471 A1
(43) Pub. Date: Apr. 10, 2014

(54) Electromechanical Measurement of Stapedius Muscle/Tendon Activity

(71) Applicant: MED-EL Elektromedizinische Geraete GmbH, Innsbruck (AT)

(72) Inventors: Roland Hessler, Innsbruck (AT); Anandhan Dhanasingh, Innsbruck (AT); Maria del Carmen Fuentes, Innsbruck (AT); Hans Wilhelm Pau, Rostock (DE); Attila Ovari, Rostock (DE); Detlef Behrend, Rostock-Warnemunde (DE); Mareike Warkentin, Rostock (DE); Olaf Specht, Rostock (DE); Wolfram Schmidt, Rostock (DE)

(73) Assignee: MED-EL Elektromedizinische Geraete GmbH, Innsbruck (AT)

(21) Appl. No.: 14/045,913

(22) Filed: Oct. 4, 2013

(30) Foreign Application Priority Data
Oct. 4, 2012 (DE) 102012218153.8

(51) Int. Cl. A61B 5/0492; A61N 1/36

(52) U.S. Cl. CPC A61B 5/0492; A61N 1/36032
USPC 600/546; 607/57

(57) ABSTRACT

A device for measuring stapedius tissue activity uses a mechanoelectrical transducer having two ends. A static end is configured for attachment to the bony pyramid in the middle ear, and a dynamic end is configured for attachment to stapedius tissue in the middle ear. The transducer generates a corresponding electrical sensing signal output when the stapedius tissue moves the dynamic end relative to the static end.

This application claims priority from German Patent Application 10 2012 218 153.8, filed Oct. 4, 2012, which is incorporated herein by reference in its entirety.

### TECHNICAL FIELD

The present invention relates to hearing prosthesis systems such as cochlear implant systems, and more specifically to measurement of stapedius tissue activity (i.e. contraction and/or stretching in the case of the stapedius muscle and movement in the case of the stapedius tendon) for such systems.

### BACKGROUND ART

Most sounds are transmitted in a normal ear as shown in FIG. 1 through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolus where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is associated with the cochlea 104, a cochlear implant with an implanted stimulation electrode can electrically stimulate auditory nerve tissue with small currents delivered by multiple electrode contacts distributed along the electrode.

FIG. 1 also shows some components of a typical cochlear implant system which includes an external microphone that provides an audio signal input to an external signal processor 111 where various signal processing schemes can be implemented. The processed signal is then converted into a digital data format, such as a sequence of data frames, for transmission into the implant 108. Besides receiving the processed audio information, the implant 108 also performs additional signal processing such as error correction, pulse formation, etc., and produces a stimulation pattern (based on the extracted audio information) that is sent through an electrode lead 109 to an implanted electrode array 110. Typically, this electrode array 110 includes multiple electrodes on its surface that provide selective stimulation of the cochlea 104.

Following surgical implantation, the cochlear implant (CI) must be custom fit to optimize its operation with the specific patient user. For the fitting process, it is important to know if an audible percept is elicited and how loud the percept is. Normally this information is gained using behavioral measures. For example, for each electrode contact the CI user is asked at what stimulation level the first audible percept is perceived (hearing threshold (THR)) and at what stimulation level the percept is too loud (maximum comfort level (MCL)). For CI users with limited auditory experiences or insufficient communication abilities (e.g., small children), these fitting parameters can be determined using objective measures.

One commonly used objective measure is the electrically evoked compound action potential (eCAP) which can be easily measured, but shows weak correlations with the MCL (r=0.57) and THR (r=0.55). See, for example, Miller et al., The Clinical Application Of Potentials Evoked From The Peripheral Auditory System, Hearing Research, 242(1-2), 184-197 (2008); incorporated herein by reference. The electrically evoked stapedius reflex threshold (eSRT) shows high correlations with the MCL. See, for example, Stephan, K. & Welzl-Müller, K., Post-Operative Stapedius Reflex Tests With Simultaneous Loudness Scaling In Patients Supplied With Cochlear Implants, Audiology, 39, 13-18 (2000) (r=0.92); and Polak, M.; Hodges, A. & Balkany, T ECAP, ESR and Subjective Levels For Two Different Nucleus 24 Electrode Arrays, Otology & Neurotology, 2005, 26, 639-645, (r=0.93-0.95); both incorporated herein by reference. But the eSRT electrical measurement is difficult to measure reliably; for example, movement artifacts of the impedance probe can introduce measurement artifacts. However, it has turned out that electromyographic (EMG) measurement of the activity of the stapedius muscle is fairly difficult for various reasons.

### SUMMARY

Embodiments of the present invention are directed to a device for measuring stapedius tissue activity (i.e., stapedius muscle and/or stapedius tendon activity) that uses a mechanoelectrical transducer having two ends. A static end is configured for attachment to the bony pyramid in the middle ear, and a dynamic end is configured for attachment to the stapedius tissue in the middle ear. The transducer generates a corresponding electrical sensing signal output when the stapedius tissue moves the dynamic end relative to the static end.

In specific embodiments, the transducer may include a mechanical strain gauge for generating the electrical sensing signal output., and there may be a substrate made of polyethylene terephthalate (PET) or polyaryletherketone (PAEK) for supporting the mechanical strain gauge. Or the transducer may use a piezoelectric foil for generating the electrical sensing signal output, and there may be a polyvinyl fluoride (PVF) foil substrate supporting the piezoelectric foil.

The transducer may form an elongated strip shape or a curved arch shape between the two ends. Or the transducer may form a curved loop shape with elongated parallel sections at the two ends that mechanically amplifies small movements of the stapedial tendon. In some embodiments at least one end may have a curved recess portion configured for attachment to the underlying anatomical structure. And some embodiments may include calibration means for isolating movement of the stapedius tissue from other movements in the middle ear of the patient.

Embodiments of the present invention also include a hearing implant fitting system and/or a hearing implant system (e.g., a cochlear implant, auditory brainstem implant, or middle ear implant) having a device according to any of the foregoing.

### BRIEF DESCRIPTION OF THE DRAWINGS

FIG. 1 shows anatomical structures of a human ear having a cochlear implant system.

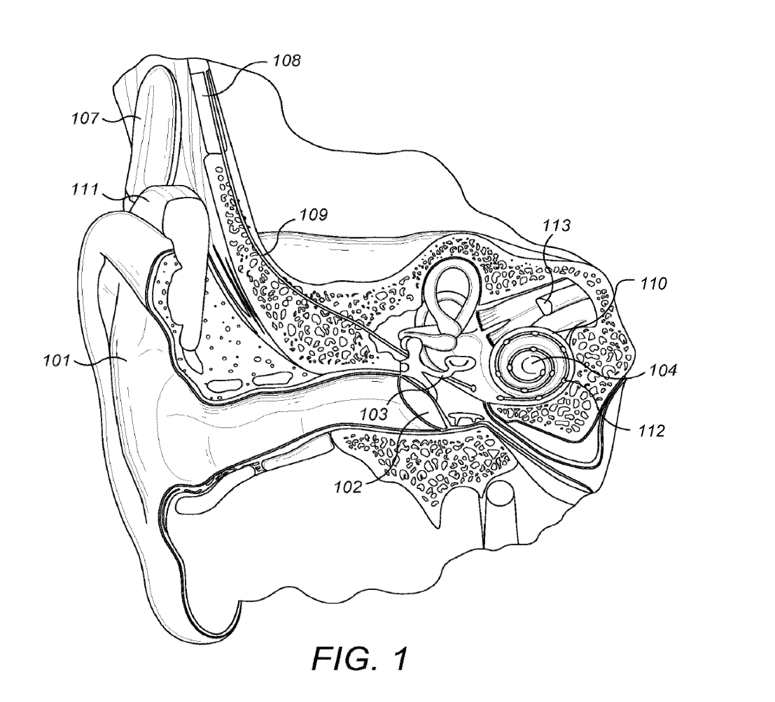

FIG. 2 shows various structural features in the middle ear with a cochlear implant electrode passing through an opening in the round window membrane.

FIG. 3 shows a mechanoelectrical transducer attached to the stapedial tendon according to one specific embodiment of the present invention.

FIG. 4 A-D shows various structural geometries of alternative embodiments.

FIG. 5 shows the structural geometry of another embodiment of the present invention.

### DETAILED DESCRIPTION

Embodiments of the present invention are direct to a mechanoelectrical transducer device for measuring stapedius tissue activity (i.e., stapedius muscle activity and/or stapedius activity), specifically, contraction of the stapedial muscle in response to loud noise. A static end of the transducer is configured for attachment to the bony pyramid in the middle ear, and a dynamic end is configured for attachment to the stapedial tendon in the middle ear. Alternatively, the dynamic end may also be attached to the stapes directly, preferable to the stapes head. The transducer device generates a corresponding electrical sensing signal output when the stapedial tendon moves the dynamic end relative to the static end.

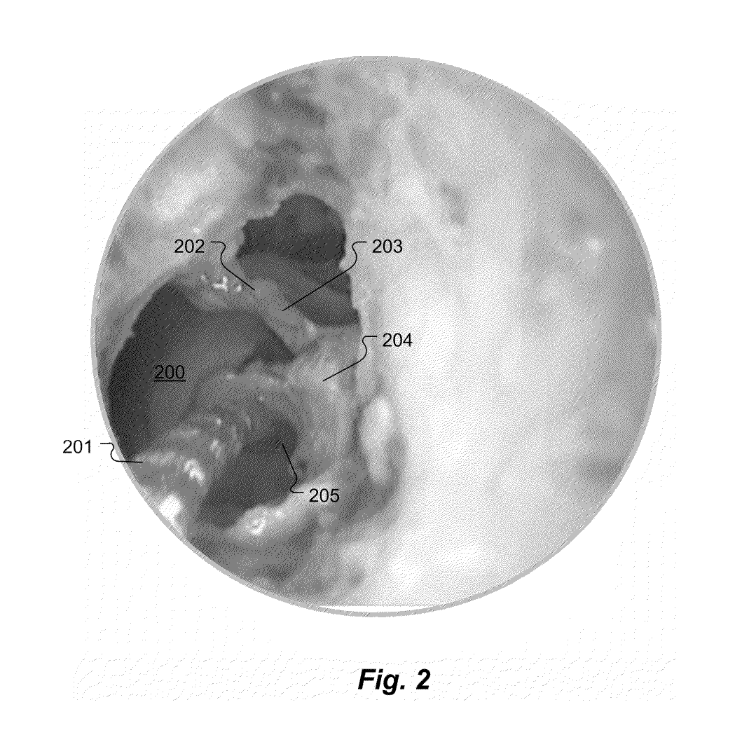

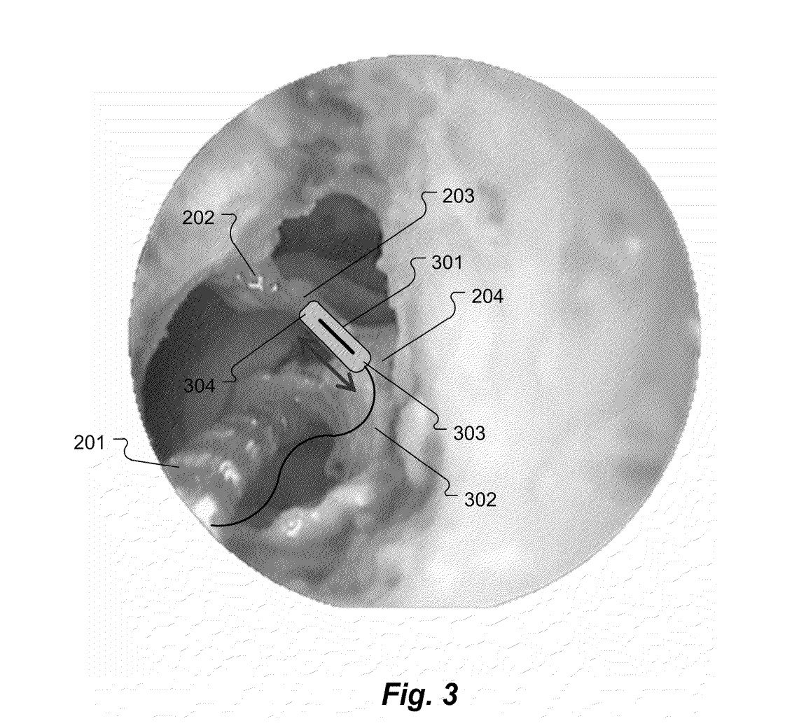

FIG. 2 shows various structural features in the middle ear 200 of a patient with a cochlear implant electrode 201 that passes through an electrode opening 205 in the round window membrane on the outer surface of the patient's cochlea. The stapedial tendon 203 is connected at one end to the stapedial muscle inside the bony pyramid 204 (pyramidal eminence) and at the other end to the stapes head 202. FIG. 3 shows that anatomical context with the addition of a mechanoelectrical transducer 301 formed in an elongated strip shape that has a static end 303 configured for attachment to the bony pyramid 204, and a dynamic end 304 configured for attachment to the stapedial tendon 203. The electrical lead 302 from the transducer 301 joins the cochlear implant electrode 201 back away from the electrode opening 205 to isolate the transducer 301 from the effects of micro-movements of the electrode 201. Alternatively rather than connecting the transducer 301 to the bony pyramid 204, in some embodiments the static end 303 of the transducer 301 may be connected to the cochlear implant electrode 201.

The transducer 301 generates a corresponding electrical sensing signal output when the stapedial tendon 203 moves the dynamic end 304 relative to the static end 303. For example, the transducer 301 may specifically be a mechanical strain gauge that changes in electrical impedance when the stapedial muscle 203 contracts in response to loud noise (e.g., MCL threshold). In such embodiments, the transducer 301 may also include a strain gauge substrate, e.g., made of polyethylene terephthalate (PET) or polyaryletherketone (PAEK), for supporting the mechanical strain gauge.

Rather than a mechanical strain gauge, some embodiments may use a transducer 301 based on a piezoelectric foil that generates an electrical signal when the dynamic end 304 moves relative to the static end 303 when the stapedial muscle 203 stretches. In such embodiments, the transducer 301 have a foil substrate that supports the piezoelectric foil; for example, made of polyvinyl fluoride (PVF).

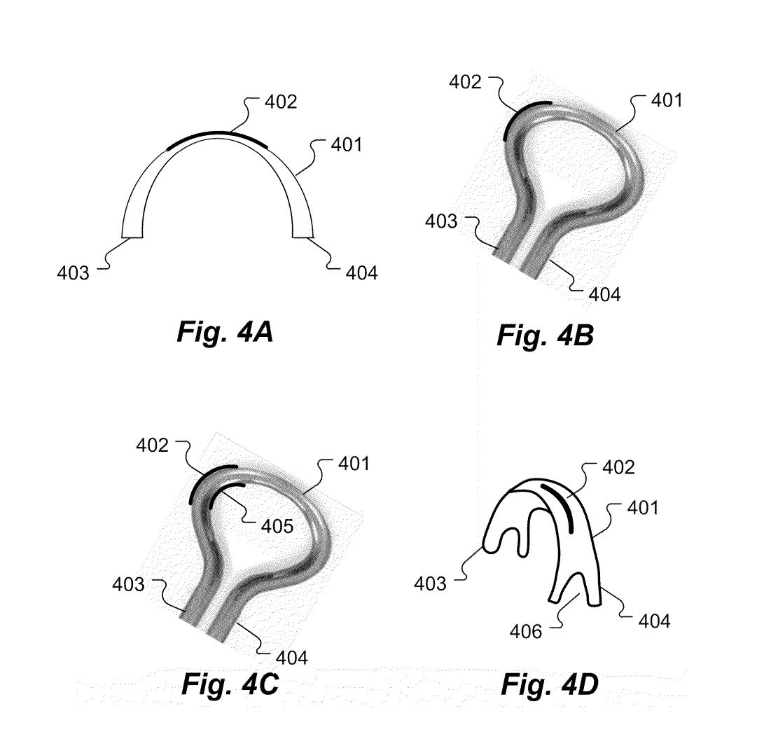

Embodiments of the present invention are not specifically limited to an elongated strip shape s shown in FIG. 3. For example, FIG. 4A shows a curved arch shape transduce 401 having a mechanical strain gauge 402 at the top of the arch between the dynamic end 403 and the static end 404. FIG. 4B shows another embodiment of a transducer 401 having a curved loop shape with elongated parallel sections at the two ends 403 and 404. The mechanical strain gauge 402 is located on the most highly curved part of the loop that is most sensitive to bending in response to small movements of the stapedial tendon 203; in effect, acting as a mechanical amplifier. FIG. 4C shows a similar embodiment with an additional strain gauge 405 on the opposite surface of the loop so that when the stapedial muscle 203 stretches in response to loud noise, one of the strain gauges 402/405 is compressed (producing increased impedance), while the other strain gauge 405/402 is stretched (producing decreased impedance).

As shown in FIG. 4D, on some transducers 401 at least one end 403 and/or 404 may have a curved recess portion 406 configured for attachment to the underlying anatomical structure—the stapedial tendon 203 and/or the bony pyramid 204. To affix an arch shaped transducer 401 such as the ones shown in FIG. 4A and 4D, the transducer 401 may use a shape memory material that is naturally biased to make the transducer 401 just slightly larger than the space where it is intended to be placed. The surgeon then compresses the ends of the transducer 401 towards each other to fit the transducer 401 in the desired location and position between the bony pyramid 204 and the stapedial tendon 203. When the surgeon releases his grip, the ends expand back out to securely fix the transducer 401 in place.

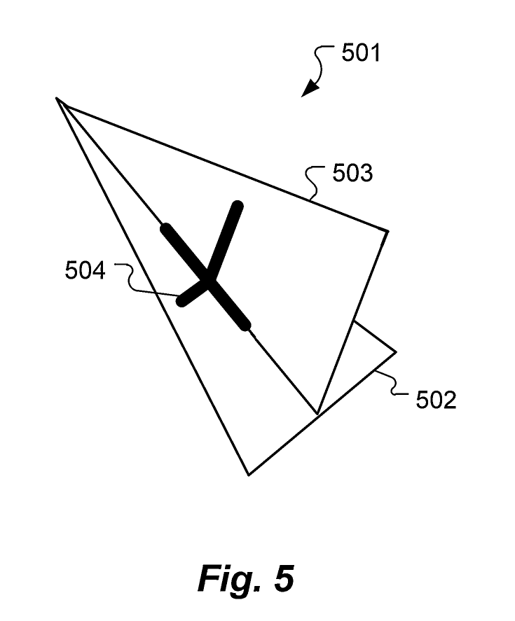

FIG. 5 shows the structural geometry of another embodiment of an electromechanical transducer 501 for the measurement of stapedius muscle activity with two perpendicular surfaces 502 and 503 with a pair of corresponding perpendicular strain gauges 504 which provide calibration means for isolating movement of the stapedial tendon from other movements in the middle ear of the patient. Specifically, when the ear is exposed to any sound, the ossicular chain in the middle ear including the stapes head moves in response. When the incoming sound is very loud, the stapedius muscle contracts and the stapedial tendon pulls on the ossicular chain to dampen the signal. That stapedius reflex response to very loud sounds is the only movement of interest for a fitting related measurement. So an embodiment such as the one shown in FIG. 5 with multiple strain gauges that are perpendicular to each other can detect movements in different spatial dimensions, and the transducer 501 thereby can be calibrated to correlate the transducer output signal with contraction of the stapedial muscle and no other movements inside the middle ear.

Although various exemplary embodiments of the invention have been disclosed, it should be apparent to those skilled in the art that various changes and modifications can be made which will achieve some of the advantages of the invention without departing from the true scope of the invention.

What is claimed is:

1. A device for measuring middle ear stapedius tissue activity comprising:
a mechanoelectrical transducer having two ends:
i. a static end configured for attachment to the bony pyramid in the middle ear of a patient, and
ii. a dynamic end configured for attachment to stapedius tissue in the middle ear of the patient;
wherein the transducer generates a corresponding electrical sensing signal output when the stapedius tissue moves the dynamic end relative to the static end.

2. A device according to claim 1, wherein the transducer includes a mechanical strain gauge for generating the electrical sensing signal output.

3. A device according to claim 2, wherein the transducer includes a polyethylene terephthalate (PET) substrate supporting the mechanical strain gauge.

4. A device according to claim 2, wherein the transducer includes a polyaryletherketone (PAEK) substrate supporting the mechanical strain gauge.

5. A device according to claim 1, wherein the transducer includes a piezoelectric foil for generating the electrical sensing signal output.

6. A device according to claim 5, wherein the transducer includes a polyvinyl fluoride (PVF) foil substrate supporting the piezoelectric foil.

7. A device according to claim 1, wherein the transducer forms an elongated strip shape between the two ends.

8. A device according to claim 1, wherein the transducer forms a curved arch shape between the two ends.

9. A device according to claim 1, wherein the transducer forms a curved loop shape with elongated parallel sections at the two ends that mechanically amplifies small movements of the stapedius tissue.

10. A device according to claim 1, wherein at least one end has a curved recess portion configured for attachment of the at least one end.

11. A device according to claim 1, further including calibration means for isolating movement of the stapedial tissue from other movements in the middle ear of the patient.

12. A hearing implant fitting system having a device according to any of claims 1-11.

13. A hearing implant system having a device according to any of claims 1-11.
